(19) United States
(12) Patent Application Publication
(10) Pub. No.: US 2025/0302740 A1
(43) Pub. Date: Oct. 2, 2025

(54) A CONTROLLED RELEASE, ANTIMICROBIAL GEL FOR THE TREATMENT OF VAGINAL INFECTIONS

(71) Applicant: SPV Healthcare Ltd., Tipperary (IE)

(72) Inventors: Stephen Costello, Dublin 15 (IE); Desmond O'Callaghan, Tipperary (IE)

(73) Assignee: SPV Healthcare Ltd., Tipperary (IE)

(21) Appl. No.: 18/864,517

(22) PCT Filed: May 18, 2022
(86) PCT No.: PCT/EP2022/063442

§ 371 Date: Nov. 9, 2024

(30) Foreign Application Priority Data
May 18, 2022 (WO) PCT/EP2022/063442

(51) Int. Cl. A61K 9/00; A61K 9/06; A61K 47/58; A61P 31/04

(52) U.S. Cl. CPC A61K 9/0034; A61K 9/06; A61K 47/58; A61P 31/04

(57) ABSTRACT

A controlled release, antimicrobial gel for the treatment of vaginal infections, comprises a muco-adhesive gel matrix containing one or more anti-infective agents bound to said matrix and effective for the treatment of vaginal infections and capable of pH dependent delivery of said one or more anti-infective agents as a single application to the vaginal cavity over a period of 3 days, while the muco-adhesive gel matrix remains bound to the vaginal epithelia. The preferred anti-infective agents are thymol and carvacrol in combination. The antimicrobial gel is biocidal against biofilms formed by the causative agents of bacterial vaginosis, trichomoniasis and candidiasis. The antimicrobial gel is effective for achieving sustained delivery of the anti-infective agents to the vaginal cavity at a therapeutically efficacious dose from a single application of the gel for the treatment and prevention, and prevention of recurrence, of bacterial vaginosis, trichomoniasis and candidiasis.

### FIELD OF THE INVENTION

This invention relates to anti-infective agents for the treatment of infections of the vaginal cavity and, in particular, to controlled/sustained release anti-infective agents for the treatment, and prevention of the recurrence, of infections of the vaginal cavity.

### BACKGROUND OF THE INVENTION

Anti-infective compositions for the treatment of infections of the vaginal cavity are known, including various types of formulations containing naturally occurring anti-infective agents.

The product Saugella Attiva, sold inter alia as a liquid (two forms of presentation, namely via a tube or a pump) and wipes for hygienic purposes, is recommended for use in situations at risk of bacterial and fungal infections, such as during the menstrual cycle, in pregnancy and in the postpartum and each contains Thymus vulgaris extract and Salvia officinalis extract as anti-infective agents. The anti-infective agents are immediately released at the site of application. The products are generally used as an adjunct to specific therapy.

Products sold under the trade mark Leucorex, are available in a number of presentations, including a gel. The products contain thymol and carvacrol as anti-infective agents. The gel releases 100% of the anti-infective agents within 24 hours, as demonstrated herein in Example 4.

The gel requires dosing twice daily and does not exhibit muco-adhesion to the wall of the vaginal cavity, such that delivery of active agent is not sustained. The quantity of anti-infective agents in the products is suited to short-term dosing.

Current first-line therapies based on either metronidazole or clotrimazole exhibit various side effects. Also, these forms of therapies do not kill biofilms and so can lead to recurrence of infection. The vaginal infections form biofilms (3-D structures of the microbes, with microbes on the surface and also below the surface). There is a difficulty with current first-line therapies in relation to killing sub-surface microbes, thus, after treatment, all microbes are not killed, and hence a recurrence of infection is frequently observed, soon after treatment finishes.

Metronidazole and clotrimazole both have low minimum inhibitory concentrations (MICs) for lactobacilli and so more readily kill the healthy lactobacilli as well as targeting infective agents. Thus, these therapies do not discriminate to the extent that is desirable between healthy and infective micro-organisms.

Side effects of metronidazole treatment include nausea, vomiting, constipation, cramping, and metallic taste.

Side effects of Clotrimazole include gastro-intestinal disorders.

Lactic acid treatment is also available over-the-counter for bacterial vaginosis; however, this treatment is not as effective as metronidazole, which is a prescription-only product.

There is a need for a formulation for the treatment of infections of the vaginal cavity which provides for controlled release of anti-infective agents for such treatment, and which prevents the recurrence of infections.

### SUMMARY OF THE INVENTION

The invention provides a controlled release, antimicrobial gel for the treatment of vaginal infections, said gel comprising a muco-adhesive gel matrix containing one or more anti-infective agents bound to said matrix and effective for the treatment of vaginal infections and capable of pH dependent delivery of said one or more anti-infective agents as a single application to the vaginal cavity over a period of 3 days, while the muco-adhesive gel matrix remains bound to the vaginal epithelia.

As used herein the terms “controlled release” and “sustained release” are used inter-changeably.

The antimicrobial gel according to the invention is effective against a range of vaginal infections including bacterial vaginosis, trichomoniasis and candidiasis or a combination thereof.

The antimicrobial gel according to the invention is effective for achieving sustained delivery of anti-infective agents to the vaginal cavity at a therapeutically efficacious dose over a 3-day period from a single application of the gel for the treatment and prevention, and prevention of recurrence, of bacterial vaginosis, trichomoniasis and candidiasis.

The antimicrobial gel according to the invention remains effective in situ for 3 days, so that only a single application every 3 days is required for effective treatment and the delivery of an effective amount continuously over 3 days. Release of antibacterial agents from the gel matrix is achievable over a period of hours and days.

Preferably, the antimicrobial gel is biocidal against biofilms formed by the causative agents of bacterial vaginosis, trichomoniasis and candidiasis.

Preferably, the release of anti-infective agent to the vaginal cavity is higher within 3 hours of application of the gel at a pH greater than 4.5, indicative of higher infection levels.

The gel according to the invention provides pH dependent release of anti-infective agent; in the first 3 hours of dosing at higher pH levels, and hence at higher infection levels, the rate of release is higher than that at lower pH levels and hence lower infection levels. Therefore, when there is a greater need for the anti-infective agents they are released faster.

According to one embodiment of the invention, up to 40% or more of anti-infective agent is released within 3 hours of application of the gel.

Preferably, the gel matrix comprises a muco-adhesive agent and a gel-forming agent.

Further, preferably, each of the muco-adhesive agent and the gel-forming agent also acts as an acidifying agent.

Preferably, the pH of the gel is in the range 2.5-4.0.

More particularly, the pH of the gel is 3.8-4.0, which is the healthy pH level for a vagina. The buffering capacity of the polymers in the gel, hereinafter described, is extensive and capable of modulating the pH to that of a healthy vagina.

Preferably, the muco-adhesive agent is a polyacrylic acid polymer.

More particularly, the muco-adhesive agent is a polycarbophil polymer.

The polycarbophil polymer is muco-adhesive and attaches to the epithelia of the vagina. The life-cycle of epithelia is 3 days with the epithelia then shed and replaced with new epithelia. When the epithelia is shed after 3 days, the residual product attached thereto is displaced and expressed from the vagina with the shed epithelia.

The polycarbophil polymer also acts as a moisturising agent.

Preferably, the gel-forming agent is a polyacrylic acid-based polymer.

More particularly, the gel-forming agent is a Carbopol polymer.

Preferably, the anti-infective agent is an anti-infective phenol compound.

Preferably, the anti-infective phenol compound is thymol or carvacrol or a combination thereof.

Thymol is a naturally-occurring monoterpenoid phenol of p-cymene, isomeric with carvacrol, and is found in oil of thyme, and extracted from Thymus vulgaris.

Thymol inhibits the adhesion of the infective agents Gardnerella vaginalis and Candida albicans to human vaginal cells.

Thymol also inhibits formation of Candida albicans and Gardnerella vaginalis bio-films, as well as killing bio-films of the infective agents.

Furthermore, thymol and carvacrol act as anti-oxidant and as anti-inflammatory agents in the vaginal canal.

Synergy and increased effectiveness are observed when thymol and carvacrol are combined compared to when each is used separately.

The effectiveness of the anti-infective agents, thymol and carvacrol, against bacterial vaginosis, trichomoniasis and candidiasis is well documented in the scientific literature.

The anti-infective agents are released from the gel matrix in accordance with the invention, in a controlled fashion at a concentration which will kill the infection. The extent of infection will also dictate the extent of rate of release, with higher infection giving rise to greater release in the first 3 hours of dosing. This release will continue for 3 days until the epithelia are shed and gel, which is bound to the epithelia, is removed from cavity. A new application of gel may then be required. The gel will kill the infective cells, although it will not kill the good bacteria (lactobacilli) of the vagina, unlike treatments with metronidazole and clotrimazole, which kill lactobacilli more readily than thymol or carvacrol.

Preferably, the antimicrobial gel contains a humectant.

A preferred product is an aqueous gel containing two anti-infective agents, a muco-adhesive polymer, a gel forming and pH balancing polymer, and moisturising agents (water, palm oil glycerides, and glycerol), as hereinafter described.

There are three main vaginal infection types, namely bacterial, fungal and protozoal, and diagnosis is by examination of cells taken from the vagina. This takes time (a number of days) and treatment can be delayed until the infection is identified. With the present invention this need for diagnosis is removed or at least does not delay treatment, as it treats all three of the infection types, while a single current therapy does not treat all three and thus, there is a risk of using the wrong treatment if the anti-infective agent is administered prior to the identification of infection. In practice, self, mis-diagnosis by the individual without a doctor being involved is prevalent in the community as over-the-counter (OTC) treatment for Candidiasis, in particular, is quite popular even though Candidiasis is not the most prolific infection; bacterial vaginosis is the most prolific infection; however, sales of treatment for this condition are significantly less and the most effective therapies are not OTC.

Preferably, at the end of 72 hours, the gel matrix is shed from the vaginal cavity as a result of the normal shedding of vaginal epithelial cells.

Preferably, the gel is delivered directly to the vaginal cavity using a single-use, pre-filled syringe tube or a repeat use syringe.

The gel can be dosed directly into the vagina. In the case of a repeat use syringe, this will be filled from an accompanying larger multi-use tube.

The method of manufacture is required to maximise the capacity of the polymers to bind the anti-infective agents, whilst also maintaining a vaginal friendly pH of between 3.5 and 4. This is achieved by using both heat and polymer neutralisation. Using neutralisation alone will result in a pH of greater than 5, which is not suitable for vaginal application.

The binding of the anti-infective agents by the polymers requires the polymer chains to be opened in the presence of the anti-infective agents, and this is achieved by either heat, neutralisation, or a combination of both.

### BRIEF DESCRIPTION OF THE DRAWINGS

FIG. 1 is a graph of gel viscosity at different mixing time points during the preparation of the gel of Example 1;

FIG. 2 is a release profile for the antimicrobial gel according to the invention at pH 4.5;

FIG. 3 is a release profile for the antimicrobial gel according to the invention at pH 5.0;

FIG. 4 is a release profile for the antimicrobial gel according to the invention at pH 5.5;

FIG. 5 is a release profile for the antimicrobial gel according to the invention at pH 6.0; and

FIG. 6 is a release profile for a product sold under the trade mark Leucorex at pH 5.5.

### DETAILED DESCRIPTION OF PREFERRED EMBODIMENTS

The invention will be further illustrated by the accompanying Examples.

### EXAMPLE 1

An antimicrobial gel according to the invention was prepared containing the following composition, as set out in Table 1

TABLE 1

| | | Concentration |
|---|---|---|
| Ingredient | Function | (% m/m) |
| Water (purified) | Solvent | 82.89 |
| | Moisturising agent | |
| Polycarbophil AA-1 | Muco-adhesive | 1.0 |
| | agent | |
| | Acidifying agent | |
| | Moisturising agent | |
| Carbopol 974 P NF | Gel forming agent | 2.0 |
| | Acidifying agent | |
| Thymol | Anti-infective agent | 0.105 |
| Carvacrol | Anti-infective agent | 0.105 |
| Glycerol | Humectant | 12.9 |
| Hydrogenated palm | Humectant | 1.0 |
| oil glycerides | | |

In the above Table concentrations are expressed in % m/m. Thus, the content of thymol and carvacrol in the gel is expressed on a mass basis. % m/m is % mass per mass (more commonly used terminology is % w/w (% weight per weight); mass is the more correct term to use.

Into 20% of the water content was added 0.25% of the Carbopol 974P (Ph. Eur.; Lubrizol Advanced Materials, Inc., Cleveland, Ohio 44141-3247) content and all of thymol (Ph. Eur.; Scharlab S.L., E08181 Sentmenat, Barcelona, Spain) and carvacrol (>98% purity; Merck KGaA, Darmstadt, Germany) content.

The resulting solution was mixed under high shear for 10 min., whilst adjusting to pH 6.0 with NaOH. The solution was mixed for a further 20 min. under low shear.

In a separate vessel to water at 60° C. was added hydrogenated palm oil glycerides (Ph. Eur.; Gattefosse SAS CS 70070 69804 Saint-Priest Cedex France) and stirred until melted.

The glycerol (Ph Eur.; Merck KGaA, Darmstadt, Germany) was added and agitated aggressively until homogenous with the water.

The Polycarbophil AA-1 (Ph. Eur.; Lubrizol Advanced Materials, Inc., Cleveland, Ohio 44141-3247) and the remaining Carbopol 974 P NF were then added and homogenised under high shear mixing until a smooth milky white appearance was achieved.

The homogenisation was stopped and the mixture stirred under low shear for 3 hours at 60° C., until maximum viscosity was attained.

The thymol, carvacrol, 20% water and 0.25% Carbopol 974P NF mixture was added to above mixture and mixed under low shear at 60° C. for 1 hour.

The mixture was removed from the vessel and allow to cool to room temperature.

A concentration of 0.105% m/m was selected for each of the thymol and carvacrol to facilitate release over 3 days.

The concentration of each agent used can be altered as required to meet safety requirements and to maximise efficacy. What we have determined to date is the release of the agents based on percentage and thus, the percentage released, can be maintained even though the actual amount of agent present can alter, once this amount is within an acceptable and comparable amount to that used in developing release profiles i.e., 0.105% m/m.

The acidifying polymers also have a role to play in fighting infection and are selected accordingly. The polymers work by reducing the pH (making it more acidic) where infection is present and thus, making the environment more hostile to infection and more agreeable for healthy microbes, for example lactobacilli.

According to the present invention, the thymol and carvacrol can fight infection when still in the gel matrix, as they will come into contact with the infective microbes when still within the matrix and so can fight them prior to release from the gel. Release of thymol and carvacrol from the gel allows them to circulate within the vaginal cavity and move to areas independent of the gel itself, thus increasing efficacy, and also helping to maximise efficaciousness of a gel dose by using the full amount of the agents within the dose.

Both thymol and carvacrol are preferred for use in the present invention so as to maximise the efficacy against both bacteria and fungi. Both agents have efficacy against both types of microbes, however thymol has superior efficacy against bacteria while carvacrol has superior efficacy against fungi.

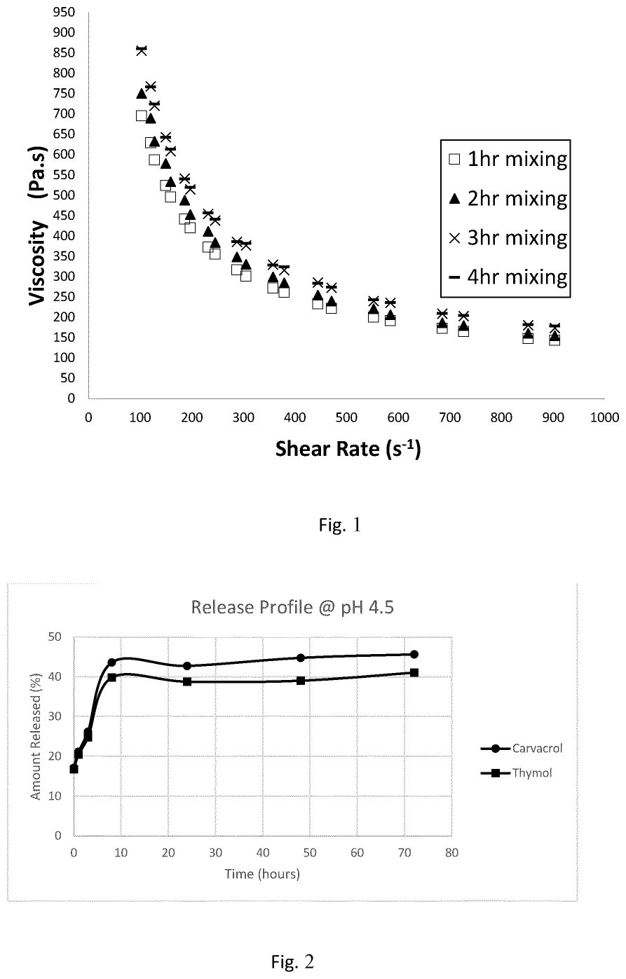

FIG. 1 is a graph of viscosity results at different mixing time points during the preparation of the gel to determine when maximum viscosity is achieved. This was achieved at 3 hours mixing, as no increase in viscosity was observed between the 3- and 4-hour time points. This shows that the thymol and carvacrol were fully encapsulated in the polymers after 3 hours of mixing.

The product thereby produced is an aqueous gel; containing 2 anti-infective agents, a muco-adhesive polymer, a gel forming and pH balancing polymer and moisturising agents, namely water, palm oil glycerides, and glycerol.

### EXAMPLE 2

A study was carried out to determine the effectiveness of thymol and carvacrol, singly or in combination, against bacterial vaginosis, trichomoniasis, candidiasis and lactobacilli relative to metronidazole, clotrimazole and clindamycin.

The results are shown in Table 2, wherein minimum inhibitory concentration (MIC) and minimum bactericidal (or fungicidal) concentration (MBC or MFC) are shown:

TABLE 2

| | Organism | | | | | |
|---|---|---|---|---|---|---|
| | Candida albicans | | Gardnerella vaginalis | | Lactobacillus | |
| | MIC | MFC | MIC | MBC | MIC | MBC |
| Agent | (μg/mL) | (μg/mL) | (μg/mL) | (μg/mL) | (μg/mL) | (μg/mL) |
| Carvacrol | 625 | 1,250 | 156 | 156 | 2,440 | 2,440 |
| Thymol | 125 | 2,500 | 175 | 250 | | |
| Thymol + Carvacrol | 625 | 625 | | | | |
| Metronidazole | | | 64 | | ≥1,000 | |
| Clotrimazole | 1,000 | ≥2,000 | | | | |
| Clindamycin | | | 4 | 8 | 1,000 | |

Gardnerella: Native biofilm was inhibited by concentrations ranging from 1 MIC to 1/8 MIC (32.77%+/−2.37 to 11.39%+/−1.46), and mature biofilm was inhibited by concentrations ranging from 1 MIC to 1/4 MIC (26.18%+/−1.36 to 13.20%+/−1.44). Thymol had been found to interfere with the adhesion of G. vaginalis to human vaginal cells (Braga PC, Dal Sasso M, Culici M, Spallino A. 2010. Inhibitory activity of thymol on native and mature Gardnerella vaginalis biofilms: in vitro study. Arzneimittelforschung 60:675-681. https://doi.org/10.1055/s-0031-1296346).

C. albicans: Thymol at 0.5xMIC showed a significant reduction (P<0.05) in biofilms formed. Thymol also has considerable effect on the destruction of mature biofilms (Braga et al 2010 supra).

Increasing the concentration of thymol was found to result in greater antimicrobial activity against infective agents, however this increasing concentration does not result in greater anti-microbial activity against

Lactobacillus acidophilus, with a steady rate of <10% inhibition of Lactobacillus acidophilus across the concentration range, compared to inhibition range of 10% to 100% across the same concentration range.

Carvacrol was found to have a minimum inhibitory concentration (MIC) of 2,440 μg/mL, which is multiple times higher than that for the infective agents, demonstrating carvacrol is more selective for the infective agents than for lactobacilli.

Synergy and increased effectiveness were observed when thymol and carvacrol were combined, compared to when each was used separately.

### EXAMPLE 3

A study was carried out from which it was determined there were residual concentrations of each anti-infective agent remaining within the gel matrix prepared in Example 1 after 72 hours. This study demonstrated the ability of the gel to bind the agents and also that the agents will remain within the gel matrix over the duration it is in situ within the vagina and thereby ensures that the gel is able to maintain efficacy itself as well as the agents which are released from it migrating throughout the vagina. Thus, treatment is attained from the gel and from the agents circulating within the cavity, which have more freedom of movement than the gel.

The results are shown in Table 3, which shows residual concentrations per each pH environment:

TABLE 3

| | |
|---|---|
| | Residual concentration post 72 hours |
| pH of | (% m/m) |

| environment | Carvacrol | Thymol |
|---|---|---|
| 4.5 | 71.15 | 67.60 |
| 5.0 | 67.33 | 63.22 |
| 5.5 | 68.36 | 64.70 |
| 6.0 | 63.23 | 57.65 |

In practice, when used in the vagina, the anti-infective agents once released will have a short period of time to remain in the vagina as they will be expressed by gravity, secretions and other factors which can remove fluid from the vagina. By having the gel bound to the epithelia, this tendency to remove fluid is negated as the bound gel will not be influenced by these factors and so there can be continual release of anti-infective agents over 72 hours.

### EXAMPLE 4

A study was carried out to determine the release profile of the antimicrobial gel prepared in Example 1 over a range of pH values relative to a gel product sold under the trade mark Leucorex, which does not have a 72-hour release profile.

To determine the concentration of active pharmaceutical ingredients (APIs) (carvacrol and thymol) in the gel
100% methanol was used as a solvent.
0.125 g of the gel of Example 1 and Leuxorex gel were weighed in 50 mL tubes.
50 mL 100% MeOH was added to both tubes (solid to solvent ratio was 1:400) and the tubes were vortexed for 3 min.
The mixture was filtered using a sterile PVDF 0.2 μm syringe filter and transferred to a vial for HPLC analysis.

Dissolution method 3× gel using various pH buffers:
Sodium-citrate buffer was prepared at four different pH values: pH 4.5, 5, 5.5 and 6
Approximately 0.25 g of gel sample was weighed in triplicate in 3 sets of 100 mL plastic containers and spread around the wall of the container in a thin layer.
100 mL of each buffer was added to one set of containers in triplicate, and 100 mL of pH 6 buffer spiked with 10.5 L carvacrol/thymol. 25 mg/mL stock solution was added to another set of containers in triplicate. Thus, solid to solvent ratio was 1:400. This method ensured no shrinking effect of API at higher levels.
The containers were covered in aluminium foil to avoid light degradation of the sample and sonicated for 5 minutes to ensure proper mixing and removal of dissolved gas from the buffer media to avoid API degradation.
Each container was placed onto an orbital shaker at 37° C. and shaken gently at 100 rpm.
1 mL aliquots were taken periodically, (0, 1, 3, 8, 24, 48 and 72 hrs.), with a sterile syringe in swirling motion, filtered with 0.2 μm sterile PVDF syringe filters followed by the direct HPLC analysis.
A standard curve of carvacrol and thymol mixture was prepared in the range of 0.5 to 7.5 μg/mL.

Chromatographic method:

The chromatographic experiment was carried out on HPLC (Agilent-1200 series) equipped with diode array detector (DAD) and Q-TOF mass spectrometer (Agilent 6520), under the following conditions:

Column-Agilent Eclipse C18 XDB, 4.6×50 mm×1.8 μm connected with XDB-4.6×1.8 μm C18 guard column.

Chromatographic separation was performed under isocratic conditions with a mobile phase composed of 2 mM ammonium formate in water-55% (Mobile Phase A) and in acetonitrile-45% (Mobile Phase B) at a flow rate of 1.0 mL/min. The column oven temperature was set to 35° C., and the auto-sampler was maintained at 4° C.

The injection volume was 10 μL with total run time of 9 minutes. Compounds of interest were detected using DAD at 195 nm as determined from previous experiments to be the optimal wavelength for the targeted compounds.

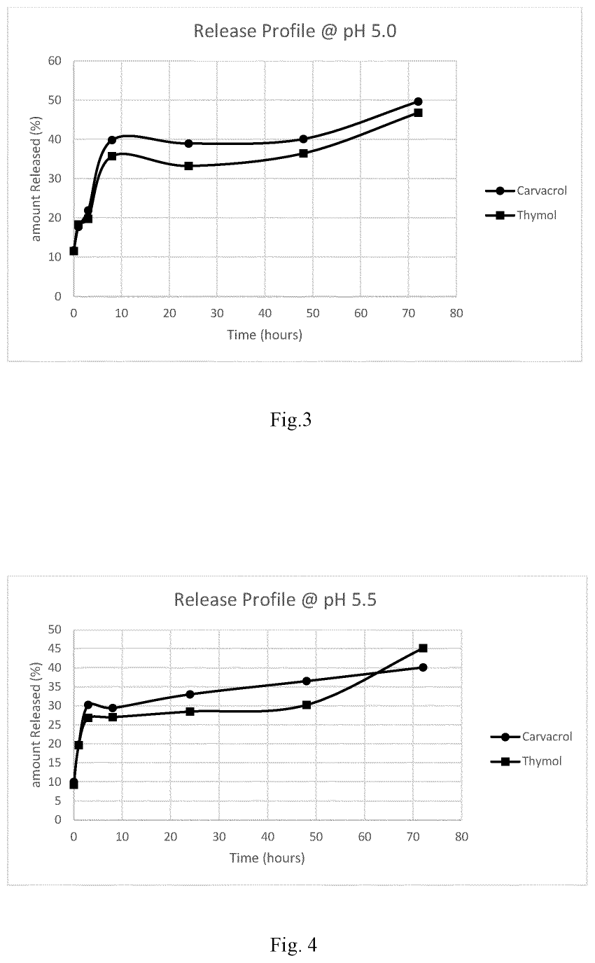

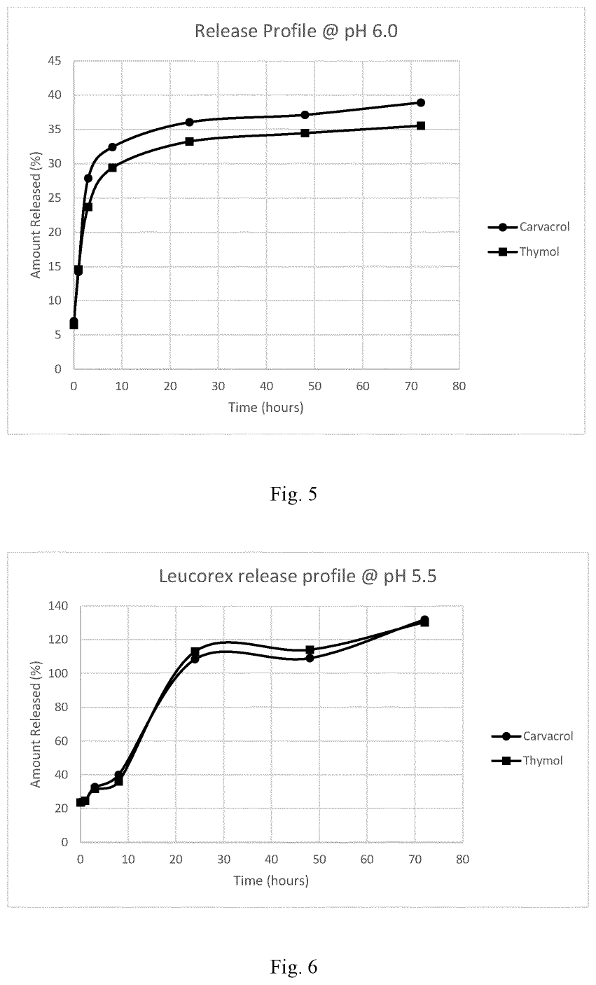

The release profiles are depicted in FIGS. 2-6.

The release profiles show i) the amount of each anti-infective agent released over 72 hours. This amount can be made equivalent to at least an efficacious amount, ii) there is always an efficacious amount of each anti-infective agent present, either released or within the gel matrix, throughout the 72 hours and iii) pH dependent release-over the first 3 hours of dosing, at higher pH and infection levels the release rate is greater than at lower pH and infection levels.

Additional advantages of the gel according to the invention are as follows:
It is selective for the bacteria which it targets; with infectious bacteria targeted and lactobacilli bacteria not targeted, thus further enabling a healthy microbial environment;
It exhibits less side effects than established therapies for bacterial vaginosis, trichomoniasis and candidiasis;
It treats and prevents infection by bacterial vaginosis, candidiasis and trichomoniasis and it treats and kills all 3 infections in a single product;
It treats vaginal atrophy;
It modulates vaginal pH to healthy levels;
It combines vaginal atrophy treatment with infection control and pH modulation; and
It has anti-inflammatory properties as thymol is known to markedly inhibit the production of TNF-α and IL-6; thus, the gel is effective at reducing inflammatory vaginitis.

As the gel according to the invention does not kill lactobacilli (healthy vaginal microbes), it enables the vagina to recover more quickly from infection, in comparison to treatment with metronidazole and clotrimazole.

The gel according to the invention has less side-effects than current first-line therapies, it kills biofilm (thereby reducing recurrence significantly). The aforementioned side effects of metronidazole and clotrimazole are not encountered with the gel according to the invention.

Antimicrobial resistance is less likely; antimicrobial resistance to metronidazole especially is growing, making this product less effective than heretofore.

Current therapies require dosing twice times a day, are not as effective at killing the infection and do not prevent recurrence.

Metronidazole is unable to inhibit or kill mature biofilm of Gardnerella vaginalis; it is only able to prevent biofilm formation. Therefore, metronidazole is not effective for killing biofilm and thus has limited effectiveness for preventing recurrence of bacterial vaginosis/Gardnerella vaginalis infection. (Gottschick C, Szafranski SP, Kunze B, Sztajer H, Masur C, Abels C, et al. (2016) Screening of Compounds against Gardnerella vaginalis Biofilms. PLOS ONE 11 (4)).

Metronidazole and clotrimazole both have lower MICs for lactobacilli and so more readily kill the healthy lactobacilli as well as targeting the infective agents than thymol or carvacrol. Thus, these therapies do not discriminate to the extent that is desirable between healthy and infective micro-organisms.

The gel according to the invention modulates pH to a healthy level and moisturises the vagina, treating vaginal atrophy. Irrespective of the type of vaginal infection the gel according to the invention can be used prior to diagnosis by examination of cells, thus saving time and treating the infection in a shorter time than current first-line therapies. It is also less expensive than current therapies.

1-17. (canceled)

18. A controlled release, antimicrobial gel for the treatment of vaginal infections, said gel comprising:
a muco-adhesive gel matrix comprising:
a muco-adhesive agent;
a gel-forming agent; and
a monoterpenoid phenol anti-infective agent bound to said matrix and fully encapsulated therein;
wherein said gel is formulated for a single application to the vaginal cavity; and
wherein a release of the anti-infective agent from the matrix is pH dependent; and
wherein the release of the anti-infective agents commences at time zero and continues releasing for a minimum of 3 days, while the muco-adhesive gel matrix remains bound to the vaginal epithelia.

19. The controlled release, antimicrobial gel of claim 18, wherein the vaginal infection is selected from bacterial vaginosis, trichomoniasis and candidiasis or a combination thereof.

20. The controlled release, antimicrobial gel of claim 19, which is biocidal against biofilms formed by the causative agents of bacterial vaginosis, trichomoniasis and candidiasis.

21. A controlled release, antimicrobial gel of claim 18, wherein the release of anti-infective agent from the gel is higher within 3 hours of application of the gel at a pH greater than 4.5, indicative of higher infection levels.

22. A controlled release, antimicrobial gel of claim 21, wherein up to 30% or more of anti-infective agent is released from the gel within 8 hours of application of the gel.

23. A controlled release, antimicrobial gel of claim 18, wherein the muco-adhesive agent and the gel-forming agent also acts as an acidifying agent.

24. A controlled release, antimicrobial gel of claim 18, wherein the pH of the gel is in the range 2.5-4.0.

25. A controlled release, antimicrobial gel of claim 18, wherein the muco-adhesive agent is a polyacrylic acid polymer.

26. A controlled release, antimicrobial gel of claim 25, wherein the muco-adhesive agent is a polycarbophil polymer.

27. A controlled release, antimicrobial gel of claim 18, wherein the gel-forming agent is a polyacrylic acid-based polymer.

28. A controlled release, antimicrobial gel of claim 27, wherein the gel-forming agent is a Carbopol polymer.

29. A controlled release, antimicrobial gel of claim 18, wherein the anti-infective monoterpenoid phenol agent is thymol or carvacrol or a combination thereof.

30. A controlled release, antimicrobial gel of claim 18, which contains a humectant.

31. A controlled release, antimicrobial gel of claim 18, wherein at the end of 72 hours, the gel matrix is shed from the vaginal cavity.

33. A controlled release, antimicrobial gel of claim 18, wherein the gel is delivered directly to the vaginal cavity using a single-use, pre-filled syringe tube.
